(19) United States
(12) Patent Application Publication
(10) Pub. No.: US 2012/0130197 A1
(43) Pub. Date: May 24, 2012

(54) QUALITY MEASUREMENTS REPORTING FOR PATIENT CARE

(75) Inventors: Andrew J. Kugler, Albany, NY (US); Suzanne A. Gunter, Concord, NC (US); John A. Lane, Weedsport, NY (US); Lari E. Shreffler, Jamesville, NY (US); Robert J. Corona, JR., Skaneateles, NY (US); Brian J. Buys, Fairport, NY (US)

(73) Assignee: WELCH ALLYN, INC., Skaneateles Falls, NY (US)

(21) Appl. No.: 13/114,070

(22) Filed: May 24, 2011

Related U.S. Application Data
(60) Provisional application No. 61/347,637, filed on May 24, 2010.

(51) Int. Cl. A61B 5/00

(52) U.S. Cl. 600/300

(57) ABSTRACT

A method for assisting a caregiver to satisfy one or more quality measures includes: selecting a quality measure; presenting, by a medical device, a first requirement associated with the quality measure to the caregiver; identifying, by the medical device, when the first requirement is satisfied; presenting a second requirement associated with the quality measure to the caregiver; identifying when the second requirement is satisfied; and selecting a billing code associated with the quality measure. Additional quality measures and/or requirements can also be addressed.

### RELATED APPLICATION

This application claims the benefit of U.S. Patent Application Ser. No. 61/347,637 filed on May 24, 2010, the entirety of which is hereby incorporated by reference.

### BACKGROUND

The Physician Quality Reporting System (PQRS), previously referred to as the Physicians Quality and Reporting Initiative (PQRI), establishes an incentive for healthcare professionals to participate in a voluntary quality reporting program. By reporting on a minimum of three measures on a specified group of patients, physicians can earn a bonus payment of on all of their Medicare billing for one year. For 2009, there were 153 quality measures and 7 measure groups in the PQRI, which can be reported to CMS (Centers for Medicare and Medicaid Services) by physicians and other caregivers in hospitals or physician practices. The number of measures can vary year to year. Other similar types of reporting initiatives also exist, such as The Joint Commission, formerly the Joint Commission on Accreditation of Healthcare Organizations (JCAHO), the National Committee for Quality Assurance (NCQA), the Agency for Healthcare Research Quality (AHRQ), the National Quality Forum (NQF), the Hospital Quality Initiative (HQI), and the American Medical Association (AMA) Physician Consortium for Performance Improvement® (PCPI). One or more of these reporting initiatives may be used in the future to increase the adoption of the most effective clinical practices through payments, penalties, and/or ratings (by payers, within a payer/provider network, etc.).

There are several methods for submitting PQRS data to CMS. These methods require the caregiver and/or staff to assure that certain measurements are taken and properly documented. The failure to do so can result in diminished care and a decrease in the reimbursements provided by programs such as Medicare and Medicaid.

### SUMMARY

Embodiments of the present disclosure are directed to systems and methods for assisting a caregiver to gather data to satisfy quality reporting programs. In examples described herein, a medical device is programmed to gather pertinent data that will assist the physician, office, etc., with meeting the quality measures for a particular patient.

One aspect is a medical device including a central processing unit (CPU) that is configured to control operation of the device; a display screen; and a set of one or more computer readable data storage media storing software instructions. The set of one or more computer readable data storage media storing software instructions, when executed by the CPU, cause the device to: identify a quality measure that is applicable to a patient; present, on the display screen, a caregiver with a textual description of at least one requirement associated with the quality measure; identify when the caregiver has collected sufficient patient data to satisfy the requirement; identify a code associated with the quality measure; and store the patient data and the code.

Another aspect is a method for assisting a caregiver to satisfy one or more quality measures. The method includes: selecting, automatically by a medical device, a quality measure; presenting, by the medical device, a first requirement associated with the quality measure to the caregiver; identifying, by the medical device, when the first requirement is satisfied; presenting a second requirement associated with the quality measure to the caregiver; identifying when the second requirement is satisfied; selecting a diagnosis associated with the quality measure; and selecting a billing code associated with the quality measure.

### DESCRIPTION OF THE DRAWINGS

The present disclosure can be better understood with reference to the description below. Within the drawings, like reference numbers are used to indicate like parts throughout the various views. Differences between like parts may cause those like parts to be each indicated by different reference numbers. Unlike parts are indicated by different reference numbers.

FIG. 1 shows block diagram illustrating an example system for collecting measurements of physiological parameters of patients.

FIG. 2 shows an example physiological monitor device.

FIG. 3 shows example physical components of the physiological monitor device of FIG. 2.

FIG. 4 shows an example graphical user interface of the device of FIG. 3.

FIG. 5 shows an example method for assisting a caregiver in satisfying quality measures.

### DETAILED DESCRIPTION

Embodiments of the present disclosure are directed to systems and methods for assisting a caregiver to gather data to satisfy quality reporting programs. Examples of such programs include the Physician Quality Reporting System (PQRS), previously referred to as the Physicians Quality and Reporting Initiative (PQRI), The Joint Commission, formerly the Joint Commission on Accreditation of Healthcare Organizations (JCAHO), the National Committee for Quality Assurance (NCQA), the Agency for Healthcare Research Quality (AHRQ), the National Quality Forum (NQF), the Hospital Quality Initiative (HQI), and the American Medical Association (AMA) Physician Consortium for Performance Improvement® (PCPI). Other quality reporting programs can also be used.

In examples described herein, a medical device is programmed to gather pertinent data that will assist the physician, office, etc., with meeting the quality measures for a particular patient.

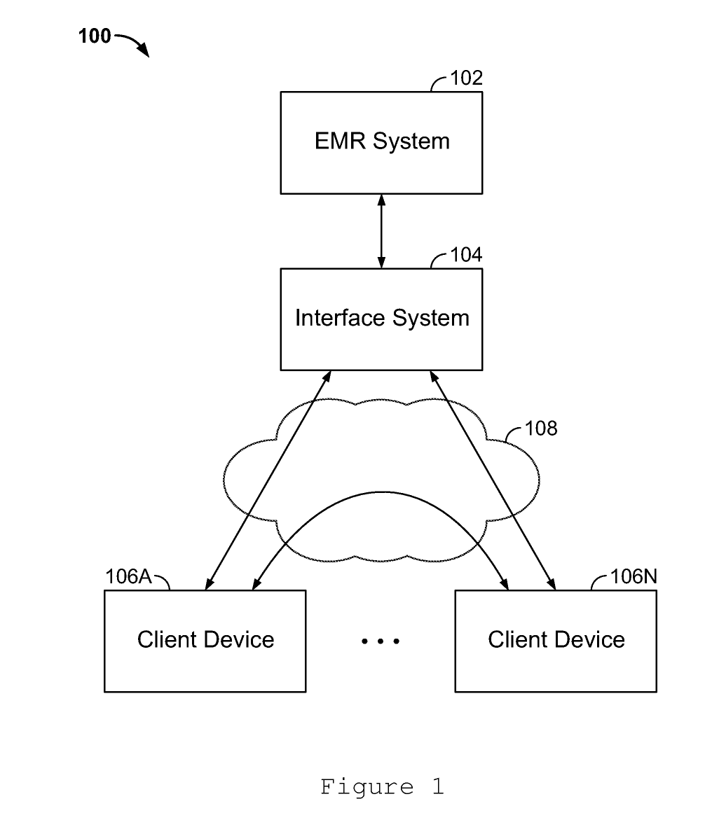

For example, FIG. 1 is a block diagram illustrating an example system 100 for collecting measurements of physiological parameters of patients. As illustrated in the example of FIG. 1, the system 100 comprises an Electronic Medical Records (EMR) system 102, an interface system 104, a set of client devices 106A-106N (collectively, “client devices 106”), and a network 108.

The network 108 is an electronic communication network that facilitates communication between the client devices 106 and the between the client devices 106 and the interface system 104. An electronic communication network is a set of computing devices and links between the computing devices. The computing devices in the network use the links to enable communication among the computing devices in the network. The network 108 can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices.

In various embodiments, the network 108 includes various types of links. For example, the network 108 can include wired and/or wireless links. Furthermore, in various embodiments, the network 108 is implemented at various scales. For example, the network 108 can be implemented as one or more local area networks (LANs), metropolitan area networks, subnets, wide area networks (such as the Internet), or can be implemented at another scale.

The EMR system 102 is a computing system that allows storage, retrieval, and manipulation of electronic medical records. As used herein, a computing system is a system of one or more computing devices. A computing device is a physical, tangible device that processes data. Example types of computing devices include personal computers, standalone server computers, blade server computers, mainframe computers, handheld computers, smart phones, special purpose computing devices, and other types of devices that process data.

Each client device in the set of client devices 106 is a computing device. The client devices 106 can provide various types of functionality. For example, the set of client devices 106 can include one or more physiological monitor devices (such as the physiological monitor device 200).

In other examples, the EMR system 102 could be replaced with a personal computing device. Data can be transferred to the personal computing device, where the data can be stored and/or forwarded. For example, in one embodiment, the data can be stored on the personal computing device and can later be forwarded electronically or by paper to a desired location, such as a billing department or reporting organization.

In addition, the set of client devices 106 can include one or more desktop, laptop, or wall-mounted devices. Such wall-mounted devices can have similar functionality to the physiological monitor device 200 but are stationary instead of portable. In addition, the set of client devices 106 can include one or more physiological monitor devices. Such monitor devices can display representations of physiological parameters. A monitor device could, for example, be used by a clinician to monitor the physiological parameters of multiple patients at one time.

The client devices 106 can communicate with each other through the network 108. In various embodiments, the client devices 106 can communicate various types of data with each other through the network 108. For example, in embodiments where the set of client devices 106 includes a set of physiological monitor devices and a monitor device, each of the physiological monitor devices can send data representing measurements of physiological parameters of patients to the monitor device. In this way, the monitor device can display representations of physiological parameters to a clinician.

The interface system 104 is a computing system that acts as an interface between the EMR system 102 and the client devices 106. In some embodiments, the interface system 104 is a CONNEX® VM interface system from Welch Allyn, Inc. of Skaneateles Falls, N.Y., although other interface systems can be used. Different EMR systems have different software interfaces.

For example, the EMR system used by two different hospitals can have two different software interfaces. The interface system 104 provides a single software interface to each of the client devices 106. The client devices 106 send requests to software interface provided by the interface system 104. When the interface system 104 receives a request from one of the client devices 106, the interface system 104 translates the request into a request that works with the software interface provided by the EMR system 102. The interface system 104 then provides the translated request to the software interface provided by the EMR system 102. When the interface system 104 receives a response from the EMR system 102, the interface system 104 translates the response from a format of the EMR system 102 to a system understood by the client devices 106. The interface system 104 then forwards the translated response to an appropriate one of the client devices 106.

The client devices 106 can send various types of data to the interface system 104 for storage in the EMR system 102 and can receive various types of data from the EMR system 102 through the interface system 104. For example, in some embodiments, the client devices 106 can send measurements of physiological parameters to the interface system 104 for storage in the EMR system 102. In another example, a monitor device can retrieve past measurements of physiological parameters of patients from the EMR system 102 through the interface system 104.

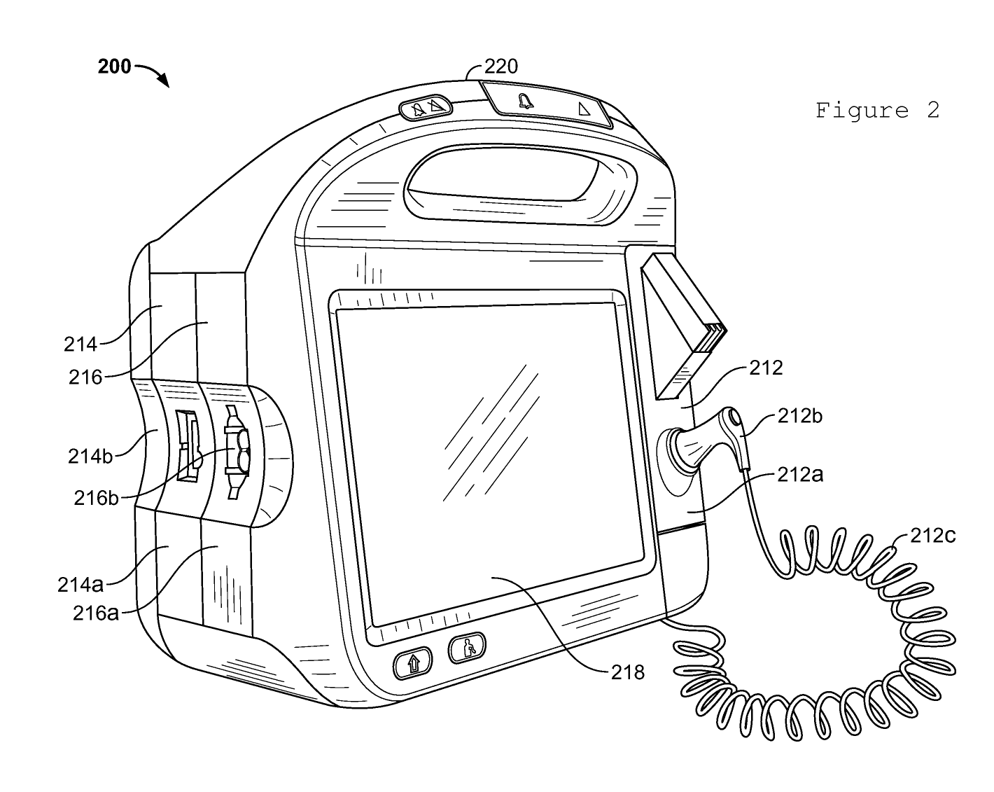

FIG. 2 illustrates a view of an example physiological monitor device 200. In this example, the physiological monitor device 200 functions as one of the client devices 106. In some examples, the physiological monitor device 200 is portable, although stationary or mounted devices can also be used. The device 200 generally assists the caregiver in gathering data associated with the patient, such as vital signs, etc. In examples, the physiological monitor device 200 may be a Connex® Vital Signs Monitor from Welch Allyn, Inc. of Skaneateles Falls, N.Y.

In the example shown, the physiological monitor device 200 includes multiple health care equipment (HCE) modules. Each of the HCE modules is configured to measure one or more physiological parameters of a health-care recipient, also referred to herein as a patient. The examples provided below are not limiting. Additional parameters can also be measured by the device 200.

A temperature measurement module 212 is accessible from the front side of the physiological monitor device 200. A SpO2 module 214 and a non-invasive blood pressure (NIBP) module 216 are accessible from a left hand side of the physiological monitor device 200. An upper handle portion 220 enables the physiological monitor device 200 to be carried by hand.

A front side of the physiological monitor device 200 includes a display screen 218 and an outer surface of the temperature measurement module 212. The temperature measurement module 212 is designed to measure the body temperature of a patient. As used in this document, a “module” is a combination of a physical module structure which typically resides within the physiological monitor device 200 and optional peripheral components (not shown) that typically attach to and reside outside of the physiological monitor device 200.

The temperature measurement module 212 includes a front panel 212a. The front panel 212a has an outer surface that is accessible from the front side of the physiological monitor device 200. The front panel 212a provides access to a wall (not shown) storing a removable probe (not shown), also referred to as a temperature probe, that is attached to a probe handle 212b. The probe and its attached probe handle 212b are tethered to the temperature measurement module 212 via an insulated conductor 212c. The probe is designed to make physical contact with a patient in order to sense a body temperature of the patient.

A left hand side of the physiological monitor device 200 includes an outer surface of the SpO2 module 214 and an outer surface of the NIBP module 216. The SpO2 module 214 is a HCE module designed to measure oxygen content within the blood of a patient. The NIBP module 216 is a HCE module designed to measure blood pressure of a patient.

As shown, the SpO2 module 214 includes a front panel 214a. The front panel 214a includes an outer surface that is accessible from the left side of the physiological monitor device 200. The front panel 214a includes a connector 214b that enables a connection between one or more peripheral SpO2 components (not shown) and a portion of the SpO2 module 214 residing inside the physiological monitor device 200. The peripheral SpO2 components reside external to the physiological monitor device 200. The peripheral SpO2 components are configured to interoperate with the SpO2 module 214 when connected to the SpO2 module 214 via the connector 214b. In some embodiments, the peripheral SpO2 components include a clip that attaches to an appendage of a patient, such as a finger. The clip is designed to detect and measure a pulse and an oxygen content of blood flowing within the patient.

As shown, the NIBP module 216 includes a front panel 216a having an outer surface that is accessible from the left side of the physiological monitor device 200. The front panel 216a includes a connector 216b that enables a connection between one or more peripheral NIBP components (not shown) and a portion of the NIBP module 216 residing inside the physiological monitor device 200. The peripheral NIBP components reside external to the physiological monitor device 200. The peripheral NIBP components are configured to interoperate with the NIBP module 216 when connected to the NIBP module 216 via the connector 216b. In some embodiments, the peripheral NIBP components include an inflatable cuff that attaches to an appendage of a patient, such as an upper arm of the patient. The inflatable cuff is designed to measure the systolic and diastolic blood pressure of the patient, the mean arterial pressure (MAP) of the patient, and the pulse rate of blood flowing within the patient.

The physiological monitor device 200 is able to operate within one or more workflows. A workflow is a series of one or more tasks that a user of the physiological monitor device 200 performs. When the physiological monitor device 200 operates within a workflow, the physiological monitor device 200 provides functionality suitable for assisting the user in performing the workflow. When the physiological monitor device 200 operates within different workflows, the physiological monitor device 200 provides different functionality.

For example, in some embodiments, the physiological monitor device 200 can operate within a monitoring workflow or a non-monitoring workflow. Example types of non-monitoring workflows include, but are not limited to, a spot check workflow and a triage workflow.

When the physiological monitor device 200 is operating within the monitoring workflow, the physiological monitor device 200 obtains a series of measurements of one or more physiological parameters of a single monitored patient over a period of time. In addition, the physiological monitor device 200 displays, on the display screen 218, a monitoring workflow home screen. The monitoring workflow home screen contains a representation of a physiological parameter of the monitored patient. The representation is based on at least one measurement in the series of measurements. A representation of a physiological parameter is a visible image conveying information about the physiological parameter.

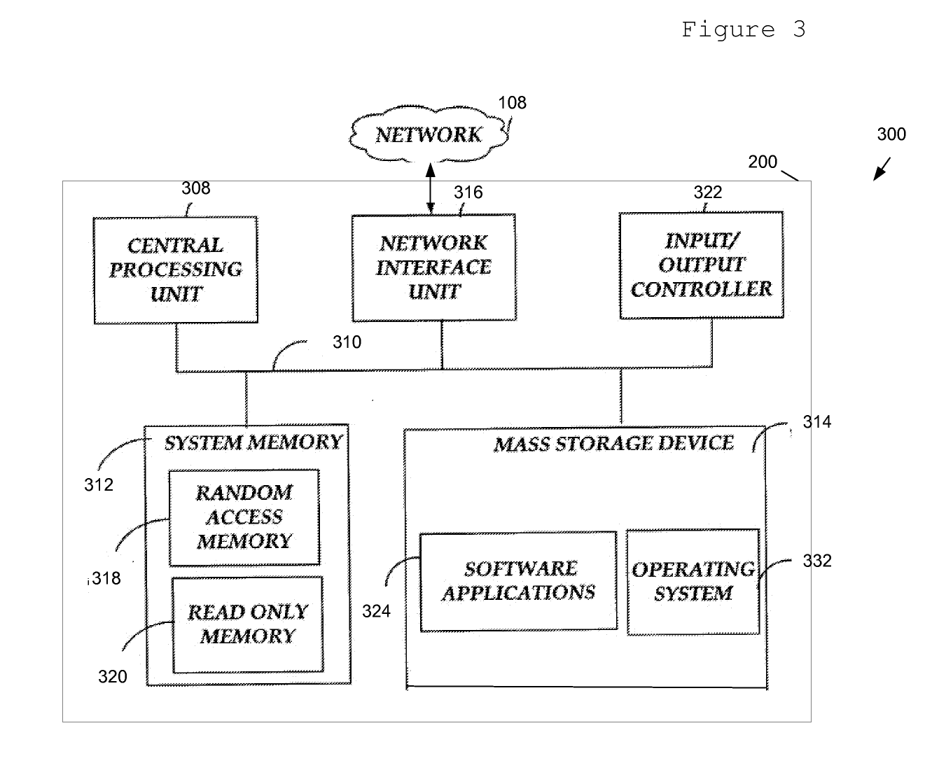

FIG. 3 illustrates example physical components of the physiological monitor device 200. As illustrated, the physiological monitor device 200 include at least one central processing unit (“CPU”) 308, a system memory 312, and a system bus 310 that couples the system memory 312 to the CPU 308. The system memory 312 includes a random access memory (“RAM”) 318 and a read-only memory (“ROM”) 320. A basic input/output system containing the basic routines that help to transfer information between elements within the physiological monitor device 200, such as during startup, is stored in the ROM 320. The physiological monitor device 200 further includes a mass storage device 314. The mass storage device 314 is able to store software instructions and data.

The mass storage device 314 is connected to the CPU 308 through a mass storage controller (not shown) connected to the bus 310. The mass storage device 314 and its associated computer-readable data storage media provide non-volatile, non-transitory storage for the physiological monitor device 200. Although the description of computer-readable data storage media contained herein refers to a mass storage device, such as a hard disk or CD-ROM drive, it should be appreciated by those skilled in the art that computer-readable data storage media can be any available non-transitory, physical device or article of manufacture from which the physiological monitor device 200 can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the physiological monitor device 200.

According to various embodiments of the invention, the physiological monitor device 200 may operate in a networked environment using logical connections to remote network devices through the network 108, such as a local network, the Internet, or another type of network. The physiological monitor device 200 connects to the network 108 through a network interface unit 316 connected to the bus 310. It should be appreciated that the network interface unit 316 may also be utilized to connect to other types of networks and remote computing systems. The physiological monitor device 200 also includes an input/output controller 322 for receiving and processing input from a number of other devices, including a keyboard, a mouse, a touch user interface display screen, or another type of input device. Similarly, the input/output controller 322 may provide output to a touch user interface display screen, a printer, or other type of output device.

As mentioned above, the mass storage device 314 and the RAM 318 of the physiological monitor device 200 can store software instructions and data. The software instructions include an operating system 332 suitable for controlling the operation of the physiological monitor device 200. The mass storage device 314 and/or the RAM 318 also store software instructions, that when executed by the CPU 308, cause the physiological monitor device 200 to provide the functionality of the physiological monitor device 200 discussed in this document. For example, the mass storage device 314 and/or the RAM 318 can store software instructions that, when executed by the CPU 308, cause the physiological monitor device to display the home screen 600 and other screens.

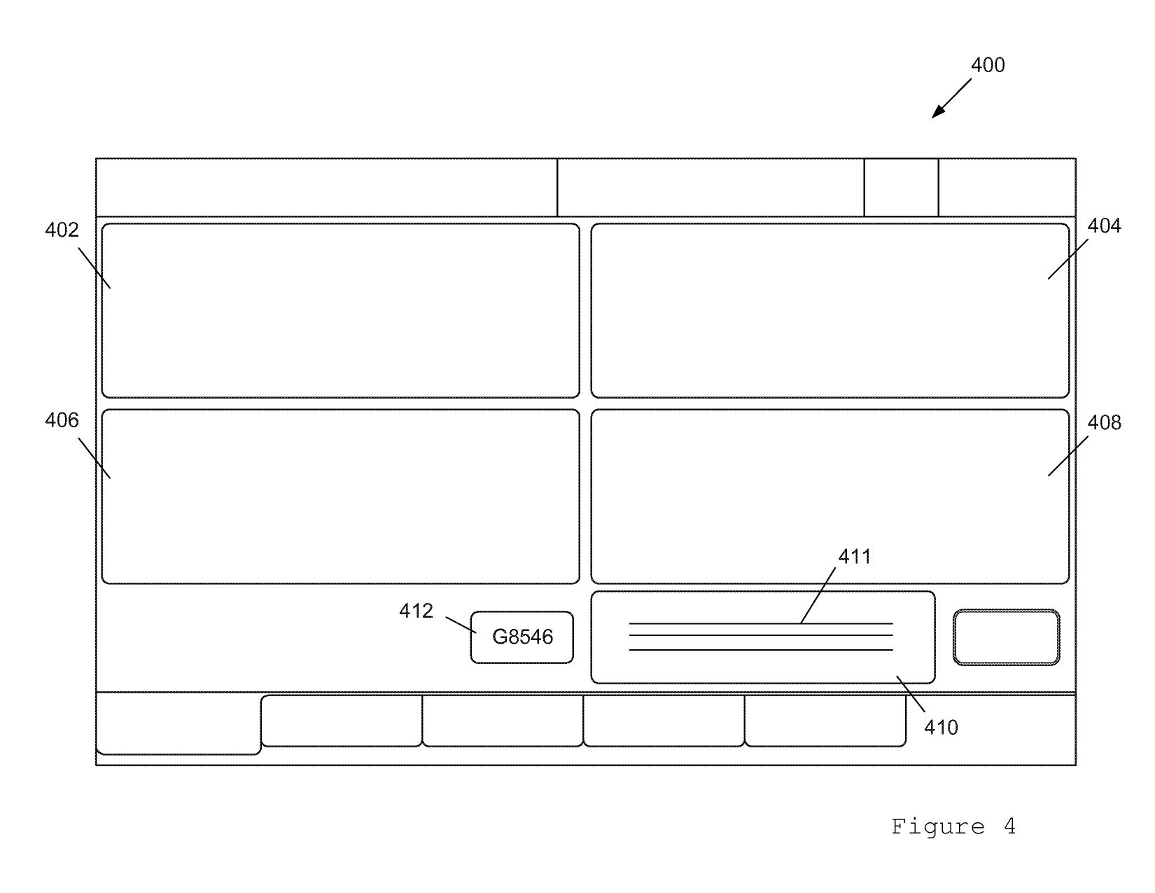

FIG. 4 illustrates an example user interface 400 displayed on the display screen 218 of the physiological monitor device 200. As described further below, the user interface 400 provides information about physiological data associated with the patient, as well as information associated with the satisfaction of one or more quality measures.

In the example shown, the user interface 400 includes a plurality of modules 402, 404, 406, 408 that show current or statistic (e.g., trended) information associated with various physiological parameters of the patient. Examples include SpO2, NIBP, temperature, pulse rate, electrocardiogram data, glucose level data, etc.

In addition, the user interface 400 includes modules 410, 412 that are programmed to provide and gather pertinent data that will assist the caregiver, office, etc., to meet the quality measures for a particular patient at the point of care. For example, in some embodiments, the device 200 is programmed to analyze data that is obtained from the patient, identify specific quality measures that are applicable to the patient data, and assist the caregiver in implementing the quality measures.

For example, the module 410 is programmed to identify quality measures that may be relevant for a patient given the physiological measurements obtained from the patient. The module 410 provides a textual display 411 that informs the caregiver if the patient is potentially eligible for a quality measure. In addition, the module 410 identifies what data outputs are needed for the fulfillment of the specific quality measure. In some examples, the module 410 also provides a description of one or more diagnoses, based on patient data, from which the caregiver can select.

For example, the module 410 can display one or more prompts based on data that has been gathered from the patient to assist the caregiver in satisfying one or more quality measures. These prompts can be displayed proactively, in that the prompts are identified as the caregiver gathers patient data and assess the patient. In addition, the prompts can be provided after the caregiver completes diagnosis of a patient, to assure that the caregiver has completed all that is required by a certain quality measure.

In some examples, the module 410 is programmed to automatically gather data that is required to meet certain measures or requirements. For example, bibliographic data associated with certain measures (e.g., gender, age, etc.), can be automatically pulled from databases, such as an EMR record, to determine qualifications for particular measures. In addition, data from other sources can also be accessed, such as physiological data from other measurement devices. In such a scenario, the physiological data can be automatically downloaded from other devices, using wired or wireless communications.

For example, there are certain quality measures associated with the Community-Acquired Pneumonia (CAP) Measure Group of the PQRI (similar requirements are provide for the PQRS). To satisfy this quality measure, the following must occur:
patient must be 18 years or older;
diagnosis of community-acquired bacterial pneumonia;
vital signs documented in medical record;
vital signs reviewed once during each unique encounter (PQRI #58); and
one of the following must occur:
   clinician documentation that vital signs were reviewed;
   dictation by the clinician including vital signs;
   clinician initials in the chart that vital signs were reviewed; or
   other indication that vital signs had been acknowledged by the clinician.
      In one example, the device 200 is programmed to provide the textual display 411 at the module 410 to assure that all of the requirements of the quality measure.

For example, when the caregiver uses the device 200 to take the vital signs (e.g., NIBP, Pulse, Respiratory Rate, SpO2, and Temperature) for the patient, the device 200 prompts the clinician to record the vitals and to document that the caregiver has reviewed them (PQRI ##56, 57). The caregiver can document review by notation in the module 410, by notation using a separate device connected to the EMR system 102, or by hand-written notes on a patient record.

Next, the module 410 prompts the caregiver to document the patient's mental status (i.e., oriented or disoriented—PQRI #58). Again, this can be documented on the device 200 or by some other method. Finally, the clinician must document whether or not the patient has an appropriate empiric antibiotic prescribed (PQRI #59).

In some examples, the module 410 automatically prompts the caregiver at each step to satisfy the quality measure. As a step is completed, the module 410 automatically moves to the next step. The caregiver can also skip steps and complete the skipped steps out of order, if desired. In another example, the module 410 simply lists all of the steps, and the caregiver can manually scroll through the steps to review them prior to completion.

Once a quality measure is complete, one or more billing codes, such as the Current Procedural Terminology (CPT) or Healthcare Common Procedure Coding System (HCPCS) codes can be associated with the quality measure to allow for credit of completion of the measure. In this example, the CPT code G8546 is associated with the patient data to allow for the CMS to provide the caregiver's credit for completion of the quality measure. The CPT code(s) at the module 412 can be sent along with the recorded patient data for storage in the EMR system 102 and subsequent billing.

The module 412 allows for the population of the specific CPT code for a quality measure. In one example, the system 100 automatically provides the code in the module 412, and the caregiver can revise the code as needed. In another example, the caregiver can manually enter a CPT code at the point of care when the patient data is collected. For example, the caregiver can enter a CPT code at the module 412, and the module 410 can present the steps necessary for the caregiver to satisfy the quality measure associated with the CPT code.

In yet another example, the caregiver can select among a list of diagnoses within the module 410 and/or the module 412. Based on this selection, the module 410 can provide the steps for any quality measures associated with the diagnosis, and the module 412 can automatically populate the proper CPT code. Other configurations are possible.

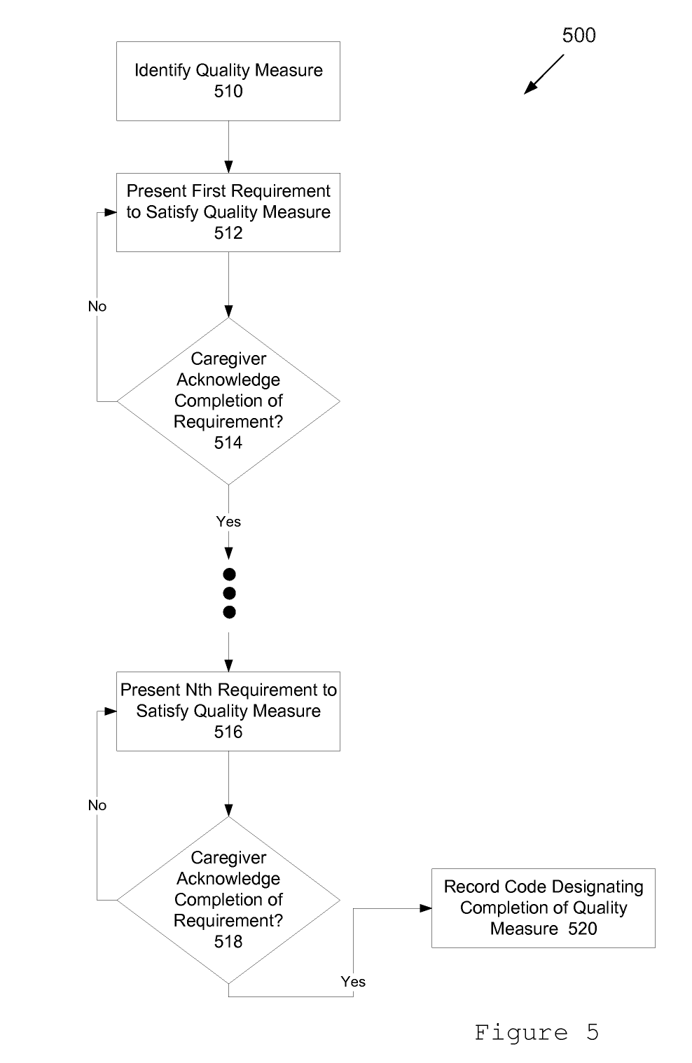

Referring now to FIG. 5, an example method for assisting a caregiver in satisfying quality measures is shown.

Initially, at operation 510, the specific quality measure or measures are identified. In one example, the caregiver can manually identify the quality measure by measure number or CPT code. In another example, the device can be programmed to automatically identify a quality measure based on the data associated with the patient. For example, if the device recognizes that the patient has elevated glucose levels, the device can suggest applying the quality measure associated with High Blood Pressure Control in Diabetes Mellitus (PQRI 2010).

Once the quality measure(s) are selected, control is passed to operation 512, and the first requirement for the measure is presented to the caregiver. For example, the device can provide a textual description of how the caregiver can satisfy the requirement (e.g., “Determine if blood pressure recording obtained within past 12 months.”).

Next, at operation 514, the device determines whether or not the caregiver has satisfied the requirement. In one example, this determination can be automatic, in that the device recognizes that the caregiver has completed the requirement based on input to the device 200, or from data acquired from an external source, such as the EMR system 102. In other examples, the caregiver can manually indicate completion of the requirement.

For example, if the requirement is to document review of a patient's vitals, the caregiver can document the review directly using the device. If done in this manner, the device can automatically determine that the requirement is met. However, if the caregiver chooses to document the review of the vitals in a hardcopy patient record, the caregiver can manually indicate that the review has been documented.

If the requirement is met, control is passed to operation 516. If not, control is passed to operation 512, and the device continues to wait for the requirement to be met. As noted previously, in some examples, the caregiver can skip requirements and complete the requirements in a different order. Also, the caregiver can cancel a particular quality measure and select a new quality measure for the device.

Next, at operation 516, the “Nth” requirement for the measure is presented to the caregiver. For example, the device can provide a textual description of how the caregiver can satisfy the requirement (e.g., “Record blood pressure.”). If the requirement is met, control is passed to operation 520. If not, control is passed to operation 516, and the device continues to wait for the requirement to be met.

Finally, at operation 520, the relevant CPT code(s) are associated with the quality measure. As noted above, the codes can be automatically provided by the device based on the quality measure, or the caregiver can manually enter the codes. Once complete, the data and codes can be sent to the EMR system for storage and billing.

Alternatives to the method 500 are possible. For example, in one alternative, the device alerts the caregiver if certain requirements are not completed. For example, if the caregiver completes a quality measure and attempts to save the data to the EMR system, the device can analyze the data to determine whether or not all requirements of the measure have been met. If not, the device provides an alert and allows the caregiver to complete the requirement(s) before the data and codes are stored in the EMR system.

In other examples, the measures can be tracked across patients to satisfy certain PQRIs/PQRSs. For example, PQRI #94 (“Otitis Media with Effusion (OME): Diagnostic Evaluation—Assessment of Tympanic Membrane Mobility”) measures the percentage of patient visits for those patients aged 2 months through 12 years with a diagnosis of OME with assessment of tympanic membrane mobility with pneumatic otoscopy or tympanometry. A similar PQRS can also be provided. The percentage is calculated with the numerator being the number of patients 2 months-12 years where TM mobility is assessed using pneumatic otoscopy or tympanometry (plus those when it wasn't and documented reasons why). The denominator is all patients in the age range diagnosed with OME. This measure advocates insufflation as primary means, with tympanometry “used to confirm the diagnosis.”

To facilitate tracking of this PQRI, the device can log each time the insufflation bulb is connected to head. The device can keep a tally each time the bulb is used. The device then can prompt the caregiver for whether or not the diagnosis is OME. For tympanometry, the device could again track the number of uses and prompt for diagnosis (or to confirm diagnosis for typical OME-shaped tympanograms). This information can be stored in the device or transmitted to the EMR for reporting.

It should be appreciated that various embodiments can be implemented (1) as a sequence of computer implemented acts or program modules running on a computing system and/or (2) as interconnected machine logic circuits or circuit modules within the computing system. The implementation is a matter of choice dependent on the performance requirements of the computing system. Accordingly, logical operations including related algorithms can be referred to variously as operations, structural devices, acts or modules. It will be recognized by one skilled in the art that these operations, structural devices, acts and modules may be implemented in software, firmware, special purpose digital logic, and any combination thereof without deviating from the spirit and scope of the present disclosure.

Although the disclosure has been described in connection with various embodiments, those of ordinary skill in the art will understand that many modifications may be made thereto. Accordingly, it is not intended that the scope of the disclosure in any way be limited by the above description.

What is claimed is:

1. A medical device comprising:
a central processing unit (CPU) that is configured to control operation of the device;
a display screen; and
a set of one or more computer readable data storage media storing software instructions that, when executed by the CPU, cause the device to:
identify a quality measure that is applicable to a patient;
present, on the display screen, a caregiver with a textual description of at least one requirement associated with the quality measure;
identify when the caregiver has collected sufficient patient data to satisfy the requirement;
identify a code associated with the quality measure; and
store the patient data and the code.

2. The device of claim 1, wherein the software instructions further cause the device to allow the caregiver to provide a diagnosis.

3. The device of claim 2, wherein the software instructions further cause the device to present one or more possible diagnoses to the caregiver.

4. The device of claim 1, wherein the software instructions further cause the device to:
present one or more possible diagnoses to the caregiver; and
allow the caregiver to select one of the diagnoses.

5. The device of claim 1, wherein the quality measure is a measure to reflect best clinical practices.

6. The device of claim 5, wherein the quality measure is associated with the Physician Quality Reporting System or the Physicians Quality and Reporting Initiative.

7. The device of claim 1, wherein the quality measure is associated with the Physician Quality Reporting System or the Physicians Quality and Reporting Initiative.

8. The device of claim 1, wherein the software instructions further cause the device to:
present a first requirement associated with the quality measure to the caregiver;
identify when the first requirement is satisfied;
present a second requirement associated with the quality measure to the caregiver; and
identify when the second requirement is satisfied.

9. The device of claim 8, wherein the software instructions further cause the device to select a billing code associated with the quality measure.

10. The device of claim 1, wherein the software instructions further cause the device to select a billing code associated with the quality measure.

11. A method for assisting a caregiver to satisfy one or more quality measures, the method comprising:
selecting a quality measure;
presenting, by a medical device, a first requirement associated with the quality measure to the caregiver;
identifying, by the medical device, when the first requirement is satisfied;
presenting a second requirement associated with the quality measure to the caregiver;
identifying when the second requirement is satisfied; and
selecting a billing code associated with the quality measure.

12. The method of claim 11, wherein selecting the quality measure further comprises allowing the medical device to automatically select the quality measure.

13. The method of claim 11, wherein selecting the quality measure further comprises allowing the caregiver to select the quality measure.

14. The method of claim 11, further comprising selecting a diagnosis associated with the quality measure.

15. The method of claim 14, further comprising allowing the medical device to automatically select the diagnosis.

16. The method of claim 11, wherein selecting the billing code further comprises allowing the medical device to automatically select the billing code.

17. The method of claim 11, wherein the quality measure is a measure to reflect best clinical practices.

18. The method of claim 17, wherein the quality measure is associated with the Physician Quality Reporting System or the Physicians Quality and Reporting Initiative.

19. The method of claim 11, wherein the quality measure is a measure to reflect best clinical practices.

20. A method for assisting a caregiver to satisfy one or more quality measures, the method comprising:
selecting, automatically by a medical device, a quality measure;
presenting, by the medical device, a first requirement associated with the quality measure to the caregiver;
identifying, by the medical device, when the first requirement is satisfied;
presenting a second requirement associated with the quality measure to the caregiver;
identifying when the second requirement is satisfied;
selecting a diagnosis associated with the quality measure; and
selecting a billing code associated with the quality measure.
